(19) United States
(12) Patent Application Publication
(10) Pub. No.: US 2025/0177261 A1
(43) Pub. Date: Jun. 5, 2025

(54) CLEANSING AGENT

(71) Applicant: Shiseido Company, Ltd., Tokyo (JP)

(72) Inventors: Makoto KURIHARA, Tokyo (JP); Rie Kurinobu, Tokyo (JP)

(73) Assignee: Shiseido Company, Ltd., Tokyo (JP)

(21) Appl. No.: 18/951,119

(22) Filed: Nov. 18, 2024

(30) Foreign Application Priority Data
Dec. 1, 2023 (JP) 2023-203843

(51) Int. Cl. A61K 8/25; A61Q 19/10

(52) U.S. Cl. CPC A61K 8/25; A61Q 19/10; A61K 2800/412; A61K 2800/5922; A61K 2800/651

(57) ABSTRACT

The present invention Provides a cleansing agent which has sufficient detergency and can achieve high stability and usability. A cleansing agent contains a smectite mineral (A), mica (B) having a hydrophilic surface, and water (C). Untreated mica is suitably used as mica having a hydrophilic surface.

### BACKGROUND OF THE INVENTION

### Field of the Invention

The present invention relates to a cleansing agent which is effective for cleansing skin and the like and has a high cleansing effect and high temperature stability.

### Background Art

Recently, development of environmentally friendly cleansing agents has come to be desired. This is because waste water after use of a synthetic cleansing agent or the like leads to environmental pollution, and therefore it is desirable to prevent such pollution. Such detergents have been studied not only for laundry or dishware/food detergents but also for body soaps, shampoos, facial cleansers, and the like.

Many environmentally friendly cleansing agents use many natural materials. This is because cleansing agents using natural materials generally have a lower environmental burden than cleansing agents using synthetic materials.

Examples of a cleansing agent using such a natural material include those containing powder such as clay minerals as an inorganic substance as a scrub agent. Such a scrub agent physically absorbs and removes sebum and sweat on the skin, and also exerts an effect of massaging the skin. For this reason, such a cleansing agent can provide an excellent feeling during use because it provides both a cleansing effect and a massage effect on the skin.

However, among the cleansing agents containing such a powder, the liquid detergent tends to cause the powder to easily settle. In order to solve such a problem, it has also been studied to increase the viscosity of the cleansing agent to obtain a so-called gel-like cleansing agent or a solid cleansing agent. A thickener is generally used for increasing the viscosity of a cleansing agent, but depending on the combination of the thickener contained in the cleansing agent and other components, a phenomenon that sufficient viscosity cannot be obtained or the viscosity decreases due to storage may occur.

For this reason, a technique of realizing a sufficient viscosity by combining other components such as powder with a cleansing agent containing a clay mineral and improving stability of the cleansing agent has also been studied, but according to the study of the present inventors, further improvement is desired.

### CITATION LIST

### Patent Literature

Patent Literature 1: JP 2022-052473 A

### SUMMARY OF THE INVENTION

In view of the above problems, an object of the present invention is to provide a cleansing agent containing a clay mineral, having sufficient detergency, and capable of achieving high stability and usability.

According to the present invention, the following invention is provided.

[1]

A cleansing agent including
a smectite mineral (A),
mica (B) having a hydrophilic surface, and
water (C).
   [2]

The cleansing agent according to [1], in which the component (B) is surface untreated mica.

[3]

The cleansing agent according to [1] or [2], in which the component (B) has an average particle size of 10 μm or more.

[4]

The cleansing agent according to any one of [1] to [3], in which the component (A) is selected from the group consisting of montmorillonite, beidellite, saponite, stevensite, and hectorite.

[5]

The cleansing agent according to any one of [1] to [4], further including an inorganic powder (D) other than the component (A) or the component (B).

[6]

The cleansing agent according to any one of [1] to [5], further including an organic powder (E).

[7]

The cleansing agent according to any one of [1] to [6], further including a moisturizing agent (F).

[8]

The cleansing agent according to any one of [1] to [7], in which the viscosity measured at 30° C. using a B-type rotational viscometer is 10,000 mPa·s or more.

[9]

The cleansing agent according to any one of [1] to [8], in which the pH at 25° C. is 7 to 12.

[10]

The cleansing agent according to any one of [1] to [9], in which a content of the component (B) is 1 to 20 mass % based on a total mass of the cleansing agent.

[11]

The cleansing agent according to any one of [1] to [10], in which the ratio B/A between the content of the component (B) and the content of the component (A) is 0.1 to 20.

According to the present invention, there is provided a cleansing agent which sufficiently exhibits detergency due to a clay mineral and exhibits excellent stability and usability. The cleansing agent according to the present invention also has an effect of improving the appearance of the skin after cleansing, and can provide brightness to the skin after cleansing.

### DETAILED DESCRIPTION OF THE INVENTION

A cleansing agent according to the present invention contains
a smectite mineral (A),
mica (B) having a hydrophilic surface, and
water (C). Each of these components and components that can be additionally blended in will be described below.

### (A) Smectite Mineral

The cleansing agent according to the invention contains smectite minerals (hereinafter, the component may be referred to as the component (A)). The smectite mineral is a kind of clay mineral.

A clay mineral is generally mainly composed of a layered silicate mineral, and is also used for applications such as cosmetics. Clay minerals classified as layered silicate minerals are further classified into 1:1 type minerals such as kaolinite minerals, 2:1 type minerals such as smectite minerals, and 2:1:1 minerals such as chlorite, and in the present invention, among these, smectite minerals are used. Furthermore, among the smectite minerals, those selected from the group consisting of montmorillonite, beidellite, saponite, stevensite, and hectorite are preferably used. Among such smectite minerals, high purity clays obtained by removing impurities from natural products or synthetic clay minerals can also be used. In addition, synthetic saponite in which a part of magnesium of saponite is substituted with aluminum is also known.

Specifically, a group of natural or synthetic montmorillonites (Kunipia (Trade name, manufactured by KUNIMINE INDUSTRIES CO., LTD.), VEEGUM (Trade name, manufactured by R. T. Vanderbilt Company, Inc.), and LAPONITE (Trade name, manufactured by BYK Japan KK.), or the like) such as montmorillonite, saponite, and hectorite can be used. Among them, synthesized smectite minerals are preferably used because in this case the effects of the present invention can be more favorably exhibited and the performance can be stabilized.

The content of the component (A) can be arbitrarily selected according to the purpose, but is preferably 0.1 to 10 mass % and more preferably 0.5 to 5 mass % based on the total mass of the cleansing agent in order to realize allowing sufficient cleanability.

(B) Mica whose Surface is Hydrophilic

The cleansing agent according to the present invention contains mica (hereinafter, may be referred to as component (B)) whose surface is hydrophilic. Mica can be selected from those generally used in cosmetics and cleansing agents and used, but the surface needs to be hydrophilic.

Known examples of mica include mica, sericite, muscovite, phlogopite, synthetic mica, red mica, biotite, lychia mica, and calcined mica, and these mica can be selected and used. In the present invention, mica is required to be hydrophilic, but since the surface of natural mica is generally hydrophilic, surface untreated mica can be used as it is. In addition, although the surface state of synthetic mica may change depending on a synthesis method or a treatment method, mica having a hydrophobic surface can also be used for the cleansing agent according to the present invention by subjecting the surface to a hydrophilic treatment. It is preferable to use mica whose surface is hydrophilic as it is, but it is also possible to perform further hydrophilic treatment for the purpose of, for example, attaching a special substituent to the surface of mica particles, or perform surface treatment to the extent that the hydrophilicity of the surface is sufficiently maintained.

In the present invention, by combining the component (B) with the component (A), it is possible to realize high stability of the cleansing agent and further exhibit an effect of improving the appearance of the skin after cleansing.

The size of the mica particles can be arbitrarily selected according to the purpose, but the average particle diameter is preferably 10 μm or more, and more preferably 15 μm or more for improving high cleanability and appearance after washing. In the present invention, the average particle diameter can be measured using, for example, a laser diffraction/scattering type particle size distribution meter, and means D50 (median diameter).

The content of the component (B) can be arbitrarily selected according to the purpose, but is preferably 1 to 20 mass % and more preferably 5 to 15 mass % based on the total mass of the cleansing agent in order to realize sufficient cleanability.

In the present invention, an excellent effect can be achieved by the interaction between the component (A) and the component (B), but the effect changes depending on the blending ratio between the component (A) and the component (B). Here, the ratio B/A between the content of the component (B) and the content of the component (A) is preferably 0.1 to 20, and more preferably 0.5 to 10 so that the effect of the present invention can be significantly exhibited.

### (C) Water

The cleansing agent according to the present invention further contains water in addition to the above components. As the water, water generally used for a cleansing agent can be used, and for example, purified water, ion-exchanged water, tap water, or the like can be used.

### (D) Inorganic Powder

The cleansing agent according to the present invention contains the above components as essential components, but may further contain an inorganic powder (hereinafter, the component may be referred to as the component (D)). The component (D) is selected from components other than the component (A) or (B). Specific examples thereof include zinc oxide, red iron oxide, talc, vermicurite, magnesium carbonate, calcium carbonate, aluminum silicate, barium silicate, calcium silicate, magnesium silicate, strontium silicate, metal tungstate, silica, zeolite, glass, barium sulfate, calcined calcium sulfate, calcium phosphate, fluorapatite, hydroxyapatite, ceramic powder, and boron nitride. An inorganic pigment of yellow iron oxide or black iron oxide can also be used as an inorganic powder. These inorganic pigments can further improve the cleansing effect by the scrubbing effect.

The content of the component (D) can be arbitrarily selected according to the purpose, but is preferably 3 to 20 mass % and more preferably 5 to 15 mass % based on the total mass of the cleansing agent in order to more strongly exhibit the effect of improving the cleanability.

### (E) Organic Powder

The cleansing agent according to the invention can further contain an organic powder (hereinafter, the component may be referred to as the component (E)). Examples of the organic powder include cellulose powder such as crystalline cellulose, polyamide resin powder such as nylon powder, polyethylene powder, polymethyl methacrylate powder, polystyrene powder, copolymer resin powder of styrene and acrylic acid, benzoguanamine resin powder, polytetrafluoroethylene powder, silicone resin powder, silk powder, wool powder, and urethane powder. The average particle diameter of such an organic powder is preferably 10 to 200 μm in order to further improve the scrubbing effect.

The content of the component (E) can be arbitrarily selected according to the purpose, but is preferably 1 to 15 mass % and more preferably 3 to 10 mass % based on the total mass of the cleansing agent in order to more strongly exhibit the effect of improving the cleanability.

### (F) Moisturizing Agent

The cleansing agent according to the present invention can further contain a moisturizing agent (hereinafter, sometimes referred to as component (F)). As the moisturizing agent, those generally used for cleansing or cosmetics can be used, but a polyhydric alcohol or a low molecular betaine compound can be used. As the polyhydric alcohol, a dihydric alcohol such as ethylene glycol, propylene glycol, or 1,3-butylene glycol, a trihydric alcohol such as glycerin, or a tetrahydric or higher alcohol can be used. As the low molecular weight betaine compound, specifically, one that forms a zwitterion with an intramolecular salt such as a quaternary ammonium base, a sulfonium base or a phosphonium base having a molecular weight of 200 or less can be used. When the molecular weight is too high, it may cause skin roughness, and thus caution is required.

When a polyhydric alcohol is used as the moisturizing agent, it is preferable to use glycerin. Furthermore, in addition to glycerin, it is preferable to further combine at least one selected from the group consisting of polyethylene glycol, 1,3-butylene glycol, and dipropylene glycol.

### (G) Other Components

The cleansing agent of the present invention may contain other optional components in addition to the above components as long as the effects of the present invention are not impaired. Examples of such optional components include components generally blended into cleansing agents, such as lower alcohols, polysaccharides, drugs, surfactants, antioxidants, ultraviolet absorbers, dyes, fragrances, and protein derivatives. In particular, lower alcohols such as ethanol are preferably used because they can impart a fresh feeling of use.

The form of use of the cleansing agent according to the present invention is not particularly limited, but the cleansing agent is excellent in feel when applied to human skin and has a high skin cleansing effect, and thus is particularly suitable as a human skin cleansing agent. In addition, the dosage form is also not limited, but is preferably a dosage form such as a gel form or a cream form having a relatively high viscosity. Specifically, the viscosity is preferably 10,000 mPa·s or more, more preferably 15,000 mPa·s or more, and particularly preferably 20,000 mPa·s or more. Here, the viscosity can be measured using a B-type rotational viscometer under the conditions of spindle number 6, a speed of rotation of 10 rpm, and 30° C.

However, the cleansing agent according to the present invention is not limited to such a dosage form or use form, and any other dosage form such as a solution form or an emulsion form may be adopted, and the use form can also be used for, for example, hair shampoo, two-in-one shampoo, body soap, and the like.

In addition, the cleansing agent according to the present invention is preferably neutral to weakly basic in order to curb irritation to the skin, and specifically, the pH at 25° C. is preferably 7 to 12.

### EXAMPLES

The present invention will be described below using examples. Note that the present invention is not limited by these examples at all. In addition, the content is mass % with respect to the total amount unless otherwise specified.

### Examples 1 to 3 and Comparative Examples 1 to 3

As shown in Table 1, each component was blended in to prepare a gel cleansing agent. The physical properties of the obtained cleansing agent were evaluated as follows.

### [Stability]

After the preparation of the cleansing agent, the state of the cleansing agent after holding at 50° C. for 30 days was visually evaluated.
a: Gel maintained
b: Gel partially liquefied
c: Gel completely liquefied
   [Appearance of Skin after Cleansing]

The appearance (brightness) of the skin after application to the skin and cleansing was visually evaluated by two judges based on Comparative Example 1 (STD).
A: Clearly brighter than STD
B: Slightly brighter than STD
C: About the same as STD
D: Slightly darker than STD
E: Obviously darker than STD

TABLE 1

| | | Comparative | | | | Comparative | Comparative |
|---|---|---|---|---|---|---|---|
| | | Example 1 | Example 1 | Example 2 | Example 3 | Example 2 | Example 3 |
| A | Bentonite | 1 | 1 | 1 | 1 | 1 | 1 |
| B | Mica A | | 10 | 20 | | | |
| | Mica B | | | | 20 | | |
| | Mica C | | | | | 10 | 20 |
| D | Talc | 5 | 5 | 5 | 5 | 5 | 5 |
| | Zinc oxide | 5 | 5 | 5 | 5 | 5 | 5 |
| E | Crystalline | 5 | 5 | 5 | 5 | 5 | 5 |
| | cellulose | | | | | | |
| F | Betaine | 1 | 1 | 1 | 1 | 1 | 1 |
| | Glycerin | 15 | 15 | 15 | 15 | 15 | 15 |
| | BG | 5 | 5 | 5 | 5 | 5 | 5 |
| | DPG | 5 | 5 | 5 | 5 | 5 | 5 |
| | Ethanol | 5 | 5 | 5 | 5 | 5 | 5 |
| C | Water | Residual | Residual | Residual | Residual | Residual | Residual |
| Evaluation | Stability | c | b | a | b | c | c |
| | Skin appearance | STD | B | A | A | B | A |
| | after cleansing | | | | | | |

In the table,

Bentonite: Kunipia G (manufactured by KUNIMINE INDUSTRIAL CO., LTD., corresponding to “montmorillonite”),

Mica A: Mica which is not surface-treated, is hydrophilic, and has an average particle diameter of 10 μm or more,

Mica B: Mica which is not surface-treated, is hydrophilic, and has an average particle diameter of less than 10 μm, and

Mica C: Mica whose surface is hydrophobic due to dimethicone treatment and has an average particle diameter of less than 10 μm

From the above results, it was found that when mica having a hydrophilic surface is combined with the smectite mineral, the cleansing agent is stabilized. When the mica having a hydrophobic surface is combined therewith, the stability is not improved. Furthermore, it was found that when the average particle diameter of mica is 10 μm or more, the improvement effect is large. In addition, it was also found that when mica is combined therewith, brightness is imparted to the skin although mica is removed after cleansing.

1. A cleansing agent comprising:
a smectite mineral (A);
mica (B) having a hydrophilic surface; and
water (C).

2. The cleansing agent according to claim 1, wherein the component (B) is surface untreated mica.

3. The cleansing agent according to claim 1, wherein the component (B) has an average particle size of 10 μm or more.

4. The cleansing agent according to claim 1, wherein the component (A) is selected from the group consisting of montmorillonite, beidellite, saponite, stevensite, and hectorite.

5. The cleansing agent according to claim 1, further comprising: an inorganic powder (D) other than the component (A) or the component (B).

6. The cleansing agent according to claim 1, further comprising: an organic powder (E).

7. The cleansing agent according to claim 1, further comprising: a moisturizing agent (F).

8. The cleansing agent according to claim 1, wherein the viscosity measured at 30° C. using a B-type rotational viscometer is 10,000 mPa·s or more.

9. The cleansing agent according to claim 1, wherein the pH at 25° C. is 7 to 12.

10. The cleansing agent according to claim 1, wherein a content of the component (B) is 1 to 20 mass % based on a total mass of the cleansing agent.

11. The cleansing agent according to claim 1, wherein the ratio B/A between the content of the component (B) and the content of the component (A) is 0.1 to 20.
